(19) United States
(12) Patent Application Publication
(10) Pub. No.: US 2008/0026116 A1
(43) Pub. Date: Jan. 31, 2008

(54) METHOD FOR PREPARING AN OXYGEN REDUCED FILM AND PACKAGE

(76) Inventors: Dexter A. Chisholm, Unionville, PA (US); Noel M. Hasty, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/781,995

(22) Filed: Jul. 24, 2007

Related U.S. Application Data
(60) Provisional application No. 60/833,729, filed on Jul. 27, 2006.

(51) Int. Cl. B65D 85/00; B65B 55/00; C12N 9/02

(52) U.S. Cl. 426/118; 435/180; 53/428

(57) ABSTRACT

The invention is directed to a method for preparing an oxygen reduced film and package by inclusion of an oxygen scavenger coating that is activable upon exposure to water vapor during final package assembly. The film includes a composition having a non-aqueous liquid vehicle, a multicopper oxidase enzyme, an oxidizable substrate and an organic binder polymer, wherein the polymer is dissolved or dispersed in the liquid vehicle, wherein the enzyme and the substrate are in particulate form and dispersed in the liquid vehicle; and wherein the liquid vehicle is evaporated.

### FIELD OF INVENTION

The invention relates to a method for preparing an oxygen reduced film and package by inclusion of an oxygen scavenger coating that is activable upon exposure to water vapor during final package assembly.

### BACKGROUND

Packaging is commonly employed for the transport, storage and display of products en route from the factory to the ultimate user. A critical function of the package is to protect the packaged item from damage or contamination during that journey. In certain applications, such as in food packaging, the package often adds various functionality, such as controlled atmosphere, microwave-heating capability and so on. There is a continuing need in the industry for cost effective methods for preparing packages with an oxygen-scavenging functionality.

Oxidative degradation of packaged goods has long been recognized as a problem affecting both appearance and useful life. Such goods include food, beverages, cosmetics, personal care products, electronic components/devices and pharmaceuticals,

Enzyme-substrate systems have long been known in the art as effective oxygen scavenging systems. Such systems include a combination of glucose oxidase and glucose; and the use of glucose oxidase, catylase, and glucose impregnated into a sheet used to wrap a packaged good. Other systems include the use of glucose oxidase, catylase, glucose and a water-soluble polymeric binder; and enzyme and substrate incorporated as a thin film between multiple layers. Ascorbate oxidase has been disclosed as an additive to ascorbate-containing foods and juices. Ascorbate oxidase in the form of an immobilized enzyme covalently bound to the inner lining of food packages has been disclosed. Laccase is an oxidase with a wide substrate range and is used as a deoxygenating food additive, where naturally occurring reducing substrates are used by laccase to convert oxygen to water.

U.S. published patent application 2005/0205840 to Farneth et al. discloses a process to remove oxygen from a sealed container wherein an O₂ scavenging system is provided comprising an enzyme and a reducing substrate. The system as described in Farneth is an easily applied water-based formulation. However, the system of Farneth actively scavenges oxygen during storage and while being applied to the container, squandering scavenging capacity and activity.

There is a need for a method for preparing an oxygen reduced package wherein a scavenger composition is utilized which remains inert during formulation, transport, storage, and application. The scavenger composition is used to make films, sheets or layers for oxygen scavenging packaging and becomes active when the composition is exposed to moisture.

### SUMMARY OF THE INVENTION

The invention is directed to a method for preparing an activable oxygen scavenging film wherein a composition is applied to a surface, wherein the composition comprises a non-aqueous liquid vehicle, a multicopper oxidase enzyme, an oxidizable substrate and an organic binder polymer, wherein the polymer is dissolved or dispersed in the liquid vehicle, wherein the enzyme and the substrate are in particulate form and dispersed in the liquid vehicle; and evaporating the liquid vehicle.

The invention is further directed to an oxygen scavenging film having a functional barrier permeable to oxygen and water vapor at a location to effectively sequester contents of a package from the activable oxygen scavenging coated surface.

The invention is still further directed to a method for preparing an oxygen reduced package by applying a composition to a surface wherein the composition comprises: a non-aqueous liquid vehicle, a multicopper oxidase enzyme, an oxidizable substrate and an organic binder polymer, wherein the polymer is dissolved or dispersed in the liquid vehicle, wherein the enzyme and the substrate are in particulate form and dispersed in the liquid vehicle; evaporating the liquid vehicle forming an activable oxygen scavenging coated surface; preparing a package having an interior and an exterior, wherein the interior of the package comprises the activable oxygen scavenging coated surface; providing a source of water vapor wherein the activable oxygen scavenging coated surface is activated; and sealing the package.

### BRIEF DESCRIPTION OF THE DRAWINGS

FIG. 1 depicts the test apparatus employed for determination of oxygen depletion rate.

FIG. 2 depicts the pattern on a flexographic printing plate.

FIG. 3 depicts a cross-sectional view of the flexographic printing plate.

### DETAILED DESCRIPTION OF THE INVENTION

For the purposes of the present invention the term “substrate” is employed according to usage in the biochemical art to refer to a redox reactive reagent. An oxidizable substrate is a reagent which will undergo oxidation. The oxidizable substrate will react with oxygen in a reaction which is catalyzed by an enzyme. The term “substrate” carries with it no reference to any particular form, shape, size, or any morphological consideration. The term “oxidizable substrate” is synonymous with the term “reductant”. The terms “film,” “sheet,” and “layer” carry their ordinary meanings, sheets being thicker than films. The term “layer” encompasses a discontinuous layer of beads or droplets as well as a continuous layer.

The phrase “diffusive oxygen contact” means the oxygen scavenger coating and the oxidatively degradeable item contained within a prepared package are in a common atmosphere so that there exists a diffusive path which an oxygen molecule may traverse between the surface of the oxidatively degradeable item and the oxygen scavenger coating. The oxygen scavenging functionality is considered useful if it reduces the oxygen concentration in the vicinity of an oxidatively degradeable item.

The present invention may be employed in the fabrication of a package of any design in the art without limitation except that the present invention requires a package sealing step. If the package is not sealed, the oxygen scavenging function imparted by the process will rapidly be overwhelmed by the unabated influx of fresh oxygen from outside the package. For the purpose of the present invention, the term “sealed” shall be understood to mean in practical terms that the rate of oxygen diffusion into the sealed package is lower than that of the unsealed package. Sealing may be effected by taping, gluing, melting, gasketing, crimping, twisting, wrapping, and any other means such as is known in the art. Accordingly, the present invention is applicable to the preparation of rigid packaging, such as that employing cardboard, metal, wood, glass, and the like. However, it extends to “flexible packaging.”

Flexible packaging has low materials cost, reduced transportation cost, lower volume, lower weight, and lower storage cost. Thin-walled molded plastic containers or melt-cast extruded thermoplastic polymer films are typically employed in flexible packaging. Molded containers may be produced by injection molding, blow molding, or thermoforming. Typical extruded films useful in flexible packaging applications are 25-75 micrometers in thickness. In any given application, the selection of the specific polymer and the specific package design will depend upon the specific requirements of the end use.

Common package types made of flexible packaging include, but are not limited to, wraps, bags, and pouches of many shapes and sizes. Packages can be formed by wrapping the food or other item included with the oxygen scavenging coating according to the present invention. Wrapping may be performed by hand or by machine. Packaging may be accomplished by inserting the item to be packaged into a preformed package with a sealable close. The preformed package will have incorporated therein the oxygen scavenging composition of the present invention either before or after insertion of the item to be packaged but prior to the sealing of the package.

Some packages are formed in a step that occurs well before the product is packaged. Then the product is placed inside the package and the package is secured. In such instances the oxygen scavenging coating may be applied to the surface of a separate package insert which is kept in the inactive state either by removing the liquid vehicle just prior to insertion in the package. Alternatively the oxygen scavenging insert may be held in a dry environment—such as a dessicator—prior to insertion in the package.

Materials suitable for use as the packaging material in the present invention are selected according to the properties needed for performance requirements. Particularly of interest are so-called barrier properties such as permeability to oxygen, CO₂, and moisture, as well as to liquids such as oils, greases, organic solvents, (e.g., gasoline), or liquid water.

Other properties of interest in package design include toughness, strength, ductility, rigidity, cut resistance, puncture resistance and low/high temperature properties. Sealability is an important property. Sealability may be characterized by heat seal and hot tack strength, heat seal and hot tack initiation temperatures, seal-through-contamination performance, caulkability, and seal integrity. Hot tack refers to the strength of the seal while still in the molten state. Caulkability refers to the ability of the sealant material to flow, filling in gaps around folds, wrinkles, or product contaminants. While it is of importance that the package be sealed, any sealing material and method of sealing commonly employed in the art is suitable for use.

Polymer materials suitable for use as the packaging material include but are not limited to linear low density polyethylene polypropylene, low density polyethylene, high density polyethylene, polyolefin plastomers, ultralow density polyethylene, metallocene-catalyzed linear low density polyethylene, enhanced polyethylene, ethylene vinyl acetate copolymers, acid copolymers and ionomers, polyamides such as nylon 6,6, nylon 6, nylon 12 and the like; polyesters such as poly(ethylene terephthalate), ethylene vinyl alcohol (EVOH), and poly(vinylidene chloride) (PVDC). These and other polymers may be laminated one to another, or combined with other materials, such as foil or paper.

Suitable polymer films include melt blown film, melt cast film or extrusion coated film on e.g., paper or aluminum foil. All of these are known in the art. Blown films are made by melting and pumping polymer through an annular die. Cast films are made by melting and pumping polymer through a flat die onto a rotating quench drum. The extrusion coating process is similar to the cast film process except that the molten polymer is coated directly onto another material. Films may also be cast from solution. Coextruded films are also suitable. Oriented films are also suitable.

Lamination is useful for combining packaging films which cannot be combined in coextrusion, such as a lamination of aluminum foil and a polyethylene film. Suitable laminations may include multiple types of polymer film, paper, and foil. Laminations can be categorized into main types: adhesive laminations and melt laminations. In adhesive laminations, the substrates are combined using an adhesive material. In extrusion laminations the substrates are adhered together using a molten polymer, often LDPE, as the adhesive layer. Laminations are highly useful for providing barrier to oxygen, moisture. The barrier functionality may be provided by foil or a high barrier polymer such as EVOH or PVDC.

Metallization is another means for achieving a high barrier packaging film, providing an excellent barrier to oxygen and water. Metallized films are used commonly for potato chip bags, nuts and salty snacks.

Packages themselves may be formed in situ as part of the packaging process, or formed in a separate process and then employed in a filling and sealing operation. Several industrial scale methods for package fabrication, filling, and sealing are known in the art. These include so called vertical form/fill/seal (VFFS), horizontal form/fill/seal (HFFS), and thermoform/fill/seal. In VFFS operations, often used for fresh produce, tubular shaped continuous film moves in a vertical direction over a filling tube. Horizontal seals are made to define the bottom and top of the bag so formed, respectively before and after filling has taken place. In HFFS operations, the film moves in a horizontal direction during the packaging step rather than vertically. Thermoform/fill/seal packaging is frequently used for bacon and processed meats.

One embodiment includes the fabrication of packaging for fresh red meat. Fresh red meat is typically transported in large pieces from the slaughtering house to local butcher shops in large bags that provide a barrier to oxygen. As well as oxygen barrier, package toughness and seal integrity are key requirements for these bags. These bags may contain PVDC or EVOH as a barrier material, along with layer of various PE resins for toughness and sealability. According to the present invention, at least a portion of the interior of the bags will be coated with the coating composition formed according to the present invention; or, alternatively there will be a separate insert comprising the coating composition suitable for the practice of the invention.

Similarly, most processed meat such as luncheon meat, ham, bologna, and salami are packaged in high oxygen barrier films. In addition to low permeability to oxygen, toughness and seal integrity are necessary to maintaining the low oxygen concentration atmosphere inside the package. According to the present invention, the interior of the package will contain the coating composition.

Cheese packaging is another application that requires low oxygen concentrations. According to the present invention, cheese is packaged in packages comprising an interior coated surface comprising the composition applied according to the present invention. Other food products as well, including fresh cut produce, require control over oxygen in the package, and are well-suited to the application of the process.

The process of the present invention may be employed to provide oxygen scavenging coatings or layers in multi-layer structures, lids, caps, labels, pads, containers and other packaging materials. In order to keep the oxygen scavenging composition from contact with the packaged food item, it is suitable to employ a multi-layer structure wherein the oxygen scavenging coating is disposed under a polymeric scavenger barrier layer. When rapid removal of oxygen from a package is desired, a polymer having high oxygen permeability is preferred for the scavenger barrier layer. In certain applications, for example when a composition comprising a polymer and oxygen scavenging composition is used as a cap liner in a packaging article, it is desirable that oxygen moves rapidly from the inside of the container, e.g. a bottle, to contact the oxygen scavenging composition. Oxygen may be present in the package as a result of the manufacturing process or in some cases it may diffuse into the package during storage.

In other applications the polymer present in the oxygen scavenging composition will act as a barrier to permeation of oxygen from outside the packaging material to inside the packaging material. In such applications it may be desirable to utilize a polymer having low oxygen permeability but high water permeability. An example of such a polymer is a copolymer of ethylene and vinyl alcohol (EVOH).

Other embodiments of the invention are laminates, by which is meant multi-layered structures. Laminates of the invention may comprise two or more layers. An oxygen scavenging composition suitable for the practice of the invention will comprise a first layer. Such compositions may include an additional polymer or polymers to form the material that comprises this first layer. As described above, the oxygen scavenging compositions may further include additional components that do not interfere with the oxygen scavenging reaction. The second layer may be formed of any material, including the same composition as the first layer. The second layer may comprise another polymeric layer, a metal layer or metal foil layer, a ceramic or glass layer, a coextrudable adhesive layer, a hot melt adhesive layer, a solvent based adhesive layer, a fabric or other porous layer, for example Tyvek® industrial packaging.

Examples of useful laminates of these types include flexible film layers of the following constructions: polypropylene/adhesive tie layer/oxygen scavenging composition suitable for the practice of the invention/adhesive tie layer/ionomeric polymer; polyethylene/adhesive tie layer/ethylene vinyl alcohol/adhesive tie layer/oxygen scavenging composition suitable for the practice of the invention/adhesive tie layer/polypropylene; metallized Mylar® polyester film/adhesive tie layer/oxygen scavenging composition suitable for the practice of the invention/adhesive tie layer/sealant layer. The first of these constructions might be useful as a lid material with the polypropylene as the outside layer. The second would be suitable for film wrap. The third would be suitable as a lid material, with the metallized Mylar® polyester film as the outside layer. A laminate structure that would be useful for bottle caps is one having an outside layer of polypropylene and a second layer of an oxygen scavenging composition suitable for the practice of the invention wherein the polymer is ethylene vinyl acetate. A three-layer laminate structure useful for bottle caps is one having aluminum as an outside layer, an oxygen scavenging composition suitable for the practice of the invention wherein the polymer is ethylene vinyl acetate as a second layer, and ethylene vinyl acetate as the inside layer.

A laminate useful as a cup or tray material is one formed of an outside layer of polypropylene, a second layer that is an adhesive tie layer, a third layer that is EVOH, a fourth layer that is an adhesive layer, a fifth that is an oxygen scavenging composition suitable for the practice of the invention wherein the polymer is polyethylene, and an inside layer that is polyethylene. Another laminate useful as a lid material is one formed of an outside layer of MYLAR® polyester film, a second layer that is a co-extrudable adhesive, a third layer that is an oxygen scavenging composition suitable for the practice of the invention wherein the polymer is polyethylene, and an inside layer that is APPEEL® lidding resin.

Typical polymers used in film layers for packaging include polypropylene, low density polyethylene, polylactic acid, polyethylene terephthalate and high density polyethylene. Tie layers formed of coextrudable adhesives such as BYNEL® are commonly used in such constructions.

The oxygen scavenging compositions of the invention and laminates of the invention can be fabricated, for example by melt processing, into shaped articles or films used in packaging applications.

In certain embodiments the compositions will scavenge oxygen in a way that removes oxygen from packaged material. In order to provide a lengthy period of oxygen scavenging activity, the scavenging composition should be separated from air outside the package by a barrier that resists permeation of oxygen. One method is to provide a thick container wall layer, for example a 10 mil thick polypropylene layer.

Alternatively, a thin layer of a polymer that has low permeability to oxygen can provide longer life for the scavenging composition. For example, ethylene vinyl alcohol copolymers (EVOH) of 0.2 mil thickness would provide the same protection as a 20 mil layer of the more permeable polypropylene. Once protected from external oxygen, the location of the oxygen scavenging layer can be varied within the package. For example, a laminate layer comprising the oxygen scavenging composition may be a separate label affixed to the inner wall of the package, a layer in the lid of a cup or tray, a bottle cap liner, or a layer of a laminated container wall. An example of the latter would be a laminate of polypropylene, Bynel® co-extrudable adhesive resin, EVOH, Bynel® co-extrudable adhesive resin, layer comprising the oxygen scavenging composition, Bynel® adhesive resin, polypropylene, with the EVOH layer located between the outside layer and the oxygen scavenging layer.

Packaged oxygen sensitive materials that may be protected using the compositions of the invention include, but are not limited to milk, yogurt, cheeses, soups, beverages such as wine, beer and fruit juices, pre-cooked meals, pharmaceuticals, and powders or materials that are difficult to treat by nitrogen purging, such as flour or noodles. In addition, the oxygen scavenging compositions of the invention and laminates of the invention protect packaged articles from the secondary effects of oxygen, such as insect damage, fungal growth, mildew and bacterial growth.

In the instant invention, the non-aqueous nature of the liquid vehicle allows the dispersed scavenger particles to remain inert until the final stage of application when the liquid vehicle is driven off and the scavenging is activated by exposure to moisture.

The present invention provides a method comprising:

contacting a surface with a coating composition to form a coated surface, the coating composition comprising a mixture of a liquid vehicle, an organic polymer, and, a particulate mixture, the particulate mixture comprising a multicopper oxidase enzyme and an oxidizable substrate;

evaporating the liquid vehicle thereby forming an activable oxygen scavenging coated surface comprising a film, sheet, or layer comprising a mixture of the polymer and the particulate mixture;

fabricating a package having an interior and an exterior, wherein the package comprises in the interior the inactive oxygen scavenging coated surface;

contacting the inactive oxygen scavenging coated surface with moisture to form an activated oxygen scavenging coated surface;

incorporating into the interior of the package an oxidatively degradeable article, in diffusive oxygen contact with the oxygen scavenging coated surface, whether or not activated;

sealing the package to reduce oxygen transport from the exterior of the package to the interior of the package.

Optionally a functional barrier may be employed at a location to effectively sequester contents of a package from the activable oxygen scavenging coated surface. The purpose of the functional barrier is to ensure that the scavenging composition is sequestered in such a manner that it does not directly contact the contents of the sealed container. The functional barrier should be permeable to O₂, such that O₂ within the headspace of the sealed container may diffuse through the functional barrier and thereby react with the O₂ scavenging composition. The package can be designed so that the activated oxygen scavenging coated surface is in diffusive oxygen contact with the food item, but is prevented from actual physical contact by virtue of compartmentalization, or a functional barrier. One such physical barrier may be an oxygen and moisture permeable film laminated to the activated oxygen scavenging film.

Typically, functional barrier materials will include polymeric materials in the form of films or matrices. Suitable polymer materials that could be used, as well as details concerning each polymer's O₂ permeabilities, are found in: 1.) “Permeability Properties of Plastics and Elastomers, 2^{nd} Ed.”, Liesl K. Massey, ed, Plastics Design Library, Norwich: N.Y. (2003); 2.) “Barrier Polymers and Structures”, Koros ed, ACS Symposium Series, American Chemical Society: Washington D.C., pp 111 & 163 (1990); and, 3.) Stannett, Poly Eng & Sci, 18(15):1129-1134 (1978). Thus, for example, a non-limiting list of polymers suitable in the present invention include: polyacrylonitrile, polymethacrylonitrile, polyvinylidene chloride, polyethylene, terephthalate, Nylon 6®, polyvinyl chloride, polyethylene, cellulose acetate, cellulose acetate butyrate, cellulose diacetate, polycarbonate, polystyrene, Neoprene®, Teflon®, poly 4-methyl pentene-1 and poly dimethyl siloxane. Of course, any polymer that is inert to the O₂ scavenging system and the contents of the sealed container, and that has sufficient permeability to O₂, can be used in the invention herein.

The functional barrier so employed may be included in the laminate structures as described above or it may be a free-standing barrier film or other configuration not itself in direct contact with the oxygen scavenging layer.

Alternatively, air itself may serve as an appropriate functional barrier when there is no possibility of direct contact between the scavenging composition and the contents of the sealed container.

One must consider the specific application in which the O₂ scavenging system is to be used, prior to selection of a specific functional barrier. For example, in some situations, a material that would only allow water vapor to pass through the material would be required to enable activation of the system, since a material that would allow liquid water to pass through the material would not be a suitable functional barrier because the O₂ scavenging composition and the contents of the sealed container could leach through a shared aqueous solution.

The present invention employs a composition comprising an organic film-forming binder polymer and a multi-copper oxidase enzyme and an oxidizable substrate wherein the multi-copper oxidase enzyme and the oxidizable substrate are in sufficiently close proximity to one another to allow oxidation of the substrate in the presence of molecular oxygen. The composition utilizes a non-aqueous liquid vehicle.

In an embodiment the multicopper oxidase enzyme and the oxidizable substrate are in particulate form wherein they are combined to form a mixture of particles of oxidizable substrate and particles of multi-copper oxidase enzyme. In another embodiment, the multicopper oxidase enzyme and the oxidizable substrate are in particulate form wherein they are a mixture of substrate particles having disposed upon the surfaces a multi-copper oxidase enzyme. In another embodiment, the multicopper oxidase enzyme and the oxidizable substrate are in particulate form wherein there is an intimate association of substrate and a multi-copper oxidase enzyme wherein the enzyme is dispersed throughout the body of the substrate particles.

The average equivalent spherical diameter of the particles of the multicopper oxidase enzyme and the oxidizable substrate as determined by light scattering techniques ranges from 1 to 100 micrometers, and preferably, 1 to 20 micrometers.

Multi-copper oxidase enzymes are suitable for use in the present invention. Examples include laccase and ascorbate oxidase.

Laccase (E.C. 1.10.3.2, Systematic Name: Benzenediol:oxygen oxidoreductase) and ascorbate oxidase (E.C. 1.10.3.3 Systematic Name: L-ascorbate:oxygen oxidoreductase) are are two classes of multi-copper oxido-reductases which perform—in combination with a suitable substrate—a four-electron reduction of molecular O₂ to form H₂O.

Laccases occur in plants, fungi, yeasts and bacteria. Best known laccase producers are fungi. Fungal laccases suitable for the purposes of the present invention herein include (but are not limited to) those isolated from Ascomycetes and Basidiomycetes. More specifically, illustrative sources of fungal laccases include those from: Aspergillus, Neurospora, Podospora, Botrytis, Collybia, Fomes, Lentinus, Pleurotus, Trametes, Rhizoctonia, Coprinus, Psaturella, Myceliophthora, Schytalidium, Polyporus, Phlebia, Coriolus, Hydrophoropsis, Agaricus, Cascellum, Crucibulum, Myrothecium, Stachybotrys, Sporormiella, Trametes versicolor, T. villosa, Myceliophthora thermophilia, Stachybotrys chartarum, Coriolus hirsutus and C. versicolor. Commercially available laccases are available from sources such as Wacker Chemie (München, Germany; T. versicolor), Novozymes (Franklinton, N.C.; M. thermophilia), Genencor (Palo Alto, Calif.; S. chartarum), Sigma-Aldrich (St. Louis, Mo.; C. versicolor) and SynectiQ (Dover, N.J.; C. hirsutus).

The source of laccase is not limiting to the invention. Thus, although fungal laccases are preferred, laccases can also be obtained from transgenic yeasts (e.g., Pichia, Saccharomyces and Kluyveromyces), transgenic fungi (e.g., Aspergillus, Trichoderma or Chrysosporium) and transgenic plants that serve as production hosts to express laccase genes cloned from other organisms (e.g., of fungal origin). Additionally, laccase may be produced from a variety of bacteria (e.g., Escherichia, Bacillus and Streptomyces).

Additionally non-native laccases may, also, be used in the invention. These modified laccases can be obtained by traditional mutagenesis (e.g., chemical, UV) or directed evolution methods (e.g., in vitro mutagenesis and selection, site-directed mutagenesis, error prone PCR, “gene shuffling”), wherein the techniques are designed to alter the amino acid sequence of the protein with the objective of improving the characteristics of the laccase. Examples of improvements would include altering substrate specificity or increasing the stability of the native enzyme.

For a general review of ascorbate oxidases, see for example: Dawson, C. R., K. G. Strothkamp and K. G. Krul. Ann N Y Acad Sci. 258:209-220 (1975)).

Ascorbate oxidases are known to originate from plants. Ascorbate oxidases suitable for the purposes of the present invention include (but are not limited to) those isolated from tobacco, soybean, cucumber, squash plants, etc. More preferred, however, are those thermally stable ascorbate oxidases that are isolated from fungi, and in particular, from species of the genus Acremonium (e.g., see U.S. Pat. No. 5,180,672).

For the purposes of the invention, suitable reducing substrates—or, synonymously, oxidizable substrates or reductants—are compounds that are capable of donating electrons to the type 1 copper site of a multi-copper oxidase, such as laccase or ascorbate oxidase. Laccase is well known to be able to accept electrons from a wide range of phenolic molecules such as flavonoids and quinones, as well as some small non-phenolic molecules. Substrates include ascorbic acid (and its salts) and isoascorbic acid (and its salts), calcium ascorbate, sodium ascorbate, and combinations thereof.

In an embodiment, the organic film-forming binder polymer is water-insoluble. The invention encompasses embodiments wherein the polymer is not soluble in the non-aqueous liquid vehicle composition. In those embodiments the composition comprises a dispersion of both the polymer and the multicopper oxidase enzyme and the oxidizable substrate in the non-aqueous liquid vehicle. In another embodiment, the composition comprises a dispersion of the multicopper oxidase enzyme and the oxidizable substrate in a solution of the polymer and non-aqueous liquid vehicle.

The discussion following is directed to embodiments wherein the polymer is soluble in the non-aqueous liquid vehicle. However, it shall be understood that the discussion applies to embodiments in which the polymer is dispersed as particles in the vehicle.

The choice of vehicle will depend largely upon the requirements of a specific application. There is no limitation on choosing the vehicle, so long as it is a liquid below the temperature of decomposition of the other components. As a practical matter, the vehicle will be selected to be compatible with the polymeric binder. Examples of vehicles include ethyl acetate, ethanol, toluene, tetrahydrofuran (THF), methyl ethyl ketone, isopropyl alcohol, dibasic esters, 2-ethyl-hexyl acetate, normal propyl acetate, n-butyl acetate, isopropyl acetate, dimethyl formamide, N-methyl pyrolodone, acetone, cyclohexane, ethylene glycol diacetate, ethyl acetate, ethanol, toluene, THF, and mixtures thereof.

In one embodiment, the coating composition is employed for food packaging, so compatibility of the vehicle and the polymer with food is important. The composition is coated onto a surface forming a film. The vehicle is evaporated. Although the vehicle is removed prior to use of the oxygen scavenging film, food contact by trace amounts of vehicle cannot be excluded as a possibility. In another embodiment, the coating composition is applied to a surface by printing. Compatibility or inertness with the printing surface is necessary.

Polymers useful in the invention are water insoluble, and may or may not be soluble in the vehicle. Polymers insoluble in the vehicle are less preferred. Water insoluble polymers impart strength and structural integrity to the resultant scavenging film, especially after long exposure to high humidity. Water insoluble polymers may exhibit significant water uptake or water permeability. In contrast, water soluble polymers become slimy and lose film strength when in a moist environment; slimy polymers may be distressing to the consumer when the composition of the invention is employed to prepare, e.g., consumer-directed packaging as in packaged meats.

Suitable polymers are either soluble or dispersible in the selected vehicle and are capable of forming a film or layer, upon deposition and evaporation of the vehicle. The particular selection of polymer will depend upon the suitability for a particular use. Polymers soluble in a vehicle are preferred. In an embodiment of the invention intended for use in food packaging, the polymer should be safe to use with food. If rapid oxygen scavenging is desired, then the polymer should be permeable to oxygen and water vapor. Examples pf polymers include but are not limited to ethylene vinyl acetate copolymers or terpolymers, such as Elvax® or Elavaloy® available from DuPont; cellulosic polymers such as cellulose acetate, cellulose acetate propionate, or cellulose acetate butyrate; acrylic polymers such as poly(butylmethacrylate) or poly(butyl methacrylate-co-methyl methacrylate); polyurethanes prepared by reacting excess aliphatic diisocyanate with polyether or polyester polyol, diamine and terminating agent; cosolvent polyamides; and polyesters such as polyethylene sebacate or poly(butylene adipate); or mixtures thereof.

The relative amounts of the ingredients of the composition will be dictated by the particular requisites of the specific end-uses. The multicopper oxidase enzyme and the oxidizable substrate are found in the composition at one part by weight of the enzyme combined with 20 to 1000, preferably 50 to 500, parts by weight of the oxidizable substrate. One part by weight of the multicopper oxidase enzyme and the oxidizable substrate is then combined with 0.05 to 20 parts by weight of the polymer in the vehicle.

As a general rule, the least amount of polymer should be employed consistent with viscosity considerations and the degree of binding necessary. For a given amount of substrate, there are advantages to higher substrate to polymer ratios. Among these are that a given volume of ink will have higher scavenging capacity, the materials cost for a given scavenger capacity is lower, polymers with limited solubility in the vehicle will still dissolve, and the polymers employed may be less permeable to oxygen or water while still permitting a useful coating.

Other additives may include hygroscopic agents, such as fructose, silica gel, or polyvinyl alcohol; plasticizers soluble in the polymer; dispersing agents such as Tween® 80, Triton® X-100 and Pluronic®; pigments, and such others that are commonly employed in the art for modifying the properties of polymers and inks. A dispersing agent helps to form a suspending medium promoting uniform and maximum separation of the fine solid particles.

In some embodiments of the invention the multicopper oxidase enzyme and the oxidizable substrate are in the form of a mixture. Any means for preparing a mixture of the multicopper oxidase enzyme and the oxidizable substrate may be employed. In an embodiment the mixture is prepared prior to combining with the vehicle. In another embodiment, the enzyme and substrate are combined in aqueous solution followed by drying and milling to produce fine particles, in the range of 1-100 micrometers in size. Spray drying is one method of producing small homogeneous particles which minimize the amount of grinding needed to achieve a desired particle size.

In another embodiment, a mixture is formed by subjecting particles of the substrate to contact with an aqueous solution of the enzyme. Many methods for performing the requisite contacting operation are known including drum coating, pan coating, fluidized bed drying, fluidized bed mixing, v-cone blending, and injector treatment methods. The substrate particles may be first ground as necessary into the 1-100 micrometer size range, prior to contacting with the enzyme solution. In the alternative, the enzyme coated particles may be subject to comminution after treatment.

In another embodiment the substrate particles are subject to milling in which the vehicle is the grinding medium. Enzyme in powder form or concentrated aqueous solution can be added during milling to produce dispersion coated substrate particles.

Alternatively, it is possible to prepare the composition of the present invention by combining the components by direct addition of each to the vehicle while mixing. The loading volumes of the active components need to be high enough so that the enzyme and reductant are in close proximity to one another in the dried film and are able to chemically react. In general, the ingredients may be combined in any order.

In an embodiment, the mixture is prepared and then combined with the vehicle. The dispersion of the mixture in the vehicle can be accomplished by using a high-speed disperser, sand mill, bead mill, or media mill. The concentration of dispersed particles in the vehicle ranges from 0.1% by weight to about 33% by weight of the total composition.

In an embodiment, a method comprises contacting a surface with a composition comprising a mixture of the vehicle, the polymer, and the multicopper oxidase enzyme and the oxidizable substrate; causing the vehicle to be evaporated thereby disposing upon a surface the composition forming a film, sheet, or layer.

In a typical use, the composition is applied to a surface, such as the inner surface of a food package, and the vehicle evaporated leaving behind a coating comprising a polymeric matrix binding the multicopper oxidase enzyme and the oxidizable substrate. In the presence of moisture or water vapor the oxygen scavenging reaction is activated. The ink or coating composition of the invention can be applied to a surface according to methods well-known in the art. Examples of suitable surfaces include: wood pulp filter paper, glass fiber filter paper, paperboard, fabric, nonwoven fabrics, polymer films, metal foils, and label stock. Examples of suitable methods of application of the composition include solution-casting, spraying, blotting, knife over roll coating, curtain coating, dip coating, metering rod coating, reverse roll coating, painting with an applicator, and printing techniques including gravure, screen, ink jet, and flexographic.

Other embodiments of the present invention form stable dispersions that make excellent printing inks. The inks can be formulated to a suitable viscosity for screen printing, gravure printing, or flexographic printing. Additives such as fillers or pigments may be incorporated without disturbing functionality.

The vehicle employed in the composition allows the compositions to be formulated well in advance of use. In some embodiments, the dispersions are stable over periods of months retaining the scavenging capacity. In the unactivated state of the unhydrated dispersed particles, the ink composition is unreactive with atmospheric oxygen which allows printing on unmodified equipment. Furthermore, the fast-drying non-aqueous vehicles enable the use of high speed printing methods.

To prepare an ink formulation, the multicopper oxidase enzyme and the oxidizable substrate are dispersed in a vehicle using a media mill, sand mill, or high speed disperser forming a dispersion. The dispersion should contain 40%-70% by weight of scavenging particles, preferably 55% to 60%; an amount of dispersant may be added equal to ½ to 1/10 the weight of scavenging particles, preferably ¼ to ⅕; and the remainder should be vehicle. Dispersion should continue until the fineness of grind measured on a Hegman gage is between 4 and 8, preferably between 6 and 8. Vehicles used in inks include ethanol, n-propanol, isopropanol, ethyl acetate, propyl acetate, toluene, hexane, dipropylene glycol monomethyl ether, dipropylene glycol monomethyl ether acetate, or mixtures thereof.

In another embodiment of ink preparation, the ink formulation is prepared by combining a vehicle and a soluble polymeric binder, and particles so that the resulting composition, based on total composition, contains 5-30% of the particles, 4.5-30% of polymer dissolved in the vehicle, and 90.5-50 wt-% vehicle. Optionally, the composition may contain plasticizer of 0 to 5% and dispersant of 0 to 8%. The ingredients can be combined in any order. Thus, the polymer may first be dissolved in the vehicle followed by addition of particulate material which is then dispersed therein; the particulate material may be in the form of dry particles or a pre-prepared particle dispersion. Alternatively, the particle dispersion may be prepared first followed by addition and dissolution of the polymer.

In another embodiment, an ink formulation is prepared by dissolving a polymeric binder in a mixture of ethyl alcohol and ethyl acetate; the weight of binder is about 90%-150% of the desired amount of the multicopper oxidase enzyme and the oxidizable substrate. To the solution a portion of the previously prepared dispersion is mixed in containing the desired weight of the multicopper oxidase enzyme and the oxidizable substrate. To the mixture may be added an amount of plasticizer equal to 1/7 to 1/15 the amount of polymeric binder, preferably ⅛- 1/10 the amount of polymeric binder; and additional vehicle to give the desired ink. The final ink composition, based on total composition, will contain 5%-30% by weight of scavenging particles, preferably 10%-15%; 5%-30% by weight of polymeric binder, preferably 10%-20%; amounts of dispersant and plasticizer based on the scavenger and binder amounts as described above; and vehicle as the remainder.

### EXAMPLES

### General Methods

Unless otherwise indicated below, all materials used in the examples were obtained from Sigma Chemical Corporation (St. Louis, Mo.).

Myceliophthora thermophilia laccase was obtained from Novozymes (Franklinton, N.C.) as DeniLite® II Base (Item #NS37008) The enzyme was supplied on an inert carrier. The enzyme represented about 2% of the total weight and was washed from the carrier using a buffer (50 mM morpholineethanesulfonic acid, pH 5.5, 1 mM ethylenediamine tetraacetic acid) to yield a solution containing 20 mg/ml enzyme.

Myceliophthora thermophilia laccase was also supplied by Novozymes in a concentrated form (NS44141) containing 95 grams of enzyme per liter of aqeuous solution, and was of sufficient purity to be used directly.

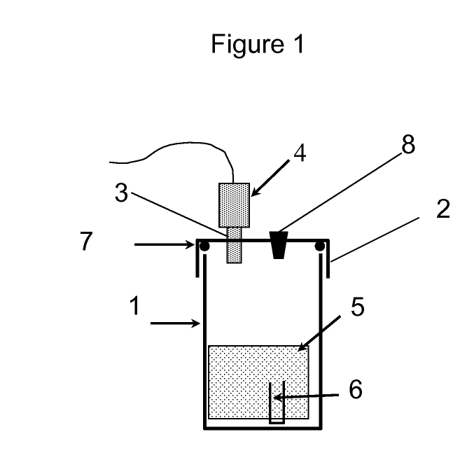

The oxygen scavenging performance of a coated film test specimen was determined by placing it in the apparatus depicted in FIG. 1 having a 20 mL scintillation vial containing a 1×6 cm strip of filter paper. A nominal 100 ml Pyrex® glass bottle 1, (actual interior volume 137 ml). was fitted with a cap 2, provided with a threaded hole, 3, drilled through the cap. An oxygen sensor 4, (Qubit Systems, s101 Diffusion Oxygen Sensor, Qubit Systems, Kingston, Ontario, Canada) was screwed through the threaded hole. The threads were sealed with epoxy. The sensor was connected to a computer (not shown) that was running Logger Pro 3.2.1 (Vernier Software and Technology). A test specimen 5, about 50 cm², was placed into the bottle before closing. Provided in the bottle was a 20 mL scintillation vial 6. The vial was used to hold Drierite® or a moist paper towel, as indicated in the specific experiments below, to avoid contact with the test specimen. A greased rubber O-ring 7, was used to seal the gap between the bottle 1 and lid 2. A rubber stopper 8, was employed for providing readily re-sealable access to the interior of the test bottle once the cap had been sealed on. The rubber stopper was removed to add water and let in oxygen after the leak test was performed.

The test specimen was placed into the jar and the jar was flushed with nitrogen to less than 1% O₂. The O₂ level was monitored for one hour to check for leaks. The rubber stopper was then removed, and 1 mL of deoxygenated water was placed in the scintillation vial. With the stopper removed, the O₂ level was allowed to rise to about 15% at 22° C. and then the rubber stopper was replaced into the hole in the cap. The O₂ content of the jar was monitored over time to determine the scavenging activity of the sample.

### Example 1

A solution of 100 g of calcium ascorbate dihydrate (Jiangsu Jiangshan Pharmaceutical Co., Ltd. Jiangshan Road, Jingjiang, Jiangsu, China) and 15 mL of Denilite concentrate (Novozymes, Franklinton, N.C.) in 1 L of nitrogen sparged water was prepared and reduced to a volume of 220 mL on a rotary evaporator. Aliquots (5 to 7 mL) were dried on a VitTis Sentry 8L freeze dry apparatus for 70 hours. The resulting solid was treated in a hammer mill (Retsch Ultra-Centrifugal Mill, ZM 200) at 18000 rpm, using a six-tooth hammer and 500 micrometer screen. The product was sieved and collected between the 10⁶ and 90 micrometer screens. The collected product was found by light scattering to have an average equivalent spherical diameter of 98 micrometers The 98-micrometer powder was passed once through an air jet mill (Fluid Energy ALJET Model: 00-JET-O-MIZER SYSTEM) and the resulting laccase/calcium ascorbate particles were found to have an average equivalent spherical diameter of 4 micrometers

A solution 0.2 g of EVA (poly(ethylene-co-vinyl acetate, Aldrich, 44% vinyl acetate) in 2.8 g of toluene was prepared. 1.0 Gram of the laccase/calcium ascorbate particles described above were added to this solution and suspended by mixing for one minute on a Vortex-Genie 2 mixer (Scientific Industries, Bohemia, N.Y.) at a speed setting of 7. A portion of this suspension was drawn down on a piece of 3-mil Mylar® polyester film (DuPont-Teijin Films) using a #60 Mayer rod. The coated area was limited to a 3×12 cm rectangle by using a stencil cut from a second piece of Mylar® film. The film with the scavenger coating was dried overnight in a vacuum oven at 21° C./22 in. Hg vacuum with a slow nitrogen purge. By weighing the film before coating and after drying, it was found that the 36 cm² block of scavenger weighed 0.1217 g, which corresponds to 33.8 g/m².

Following the test procedure for oxygen scavenging performance, it was found that over 24 hours the O₂ level dropped from 15.5 to 8.5%.

### Example 2

A mixture of 68.4 g of polysorbate 80 dispersing aid (polyoxyethylene (20) sorbitan monooleate—Tween 80K from Uniqema) and 159.6 g of absolute ethanol was prepared with mixing at 500 RPM. To this mixture was added 342.0 g of the laccase/calcium ascorbate particles described in Example 1, and the combination was mixed at 1600 rpm for 1 hour. The resulting mixture was added to a beaker with a nylon grinding disc and 0.8-1.0 mm zirconia media, and milled at 1500 rpm for 44 minutes, after which the dispersion was poured through a strainer to remove the media.

Seventy five grams of CAP 482-0.5 cellulose acetate proprionate (Eastman), 100 g of ethyl acetate and 100 g of ethanol were mixed using an air driven agitator (Fawcett LD103, Fawcett Co., Richfield, Ohio) at 500 RPM for 30 minutes. Then 134.3 g of the laccase/calcium ascorbate dispersion prepared above was added and mixing continued for 30 minutes. Then 5.0 g of glycerol triacetate plasticizer (Triacetin, Eastman), and 85.7 g of ethanol were added and mixing continued an additional 15 minutes. This gave an ink which showed no separation after standing for one week at room temperature.

### Example 3

A mixture of 550 g of Tween 80K and 930.77 g of absolute ethanol was prepared with mixing at 1000 RPM in a high speed disperser (Hockmeyer model 2L, Hockmeyer Equipment Corporation, Harrison, N.J.) with type F Cowles blade. To this mixture was added 2750 g of laccase/calcium ascorbate particles described in Example 1, and the combination was mixed at 3500 rpm for 2 hours. The resulting dispersion was let down with an additional 769.23 g of ethanol while mixing continued at 1000 RPM.

Twelve hundred grams of CAP 482-0.5 cellulose acetate proprionate (Eastman), 1600 g of ethyl acetate and 2000 g of ethanol were combined in a DBI mixer and mixed at slow speed for 30 minutes. Then 2400 g of the laccase/calcium ascorbate dispersion prepared above was added and mixing continued for 60 minutes. Then 80.0 g of triacetin (Eastman), and 720 g of ethanol were added and mixing continued an additional 20 minutes.

### Example 4

The oxygen scavenger ink prepared in Example 2 was used in a flexographic press to print a block of oxygen scavenger on a plastic film. The press was a 10″ Mark Andy Inc system 2200 flexographic printing press with 8 print stations. Only stations one and two, provided with Cyrel® (DuPont) flexographic printing plates, were used to apply two impressions of the scavenger ink. The printing stations were fitted with a 22 BCM (billion cubic micrometers/square inch) and a 33 BCM anilox roll, respectively. The oxygen scavenging coating composition was printed onto biaxially oriented polypropylene film at a speed of 100 ft/min (30.5 m/min).

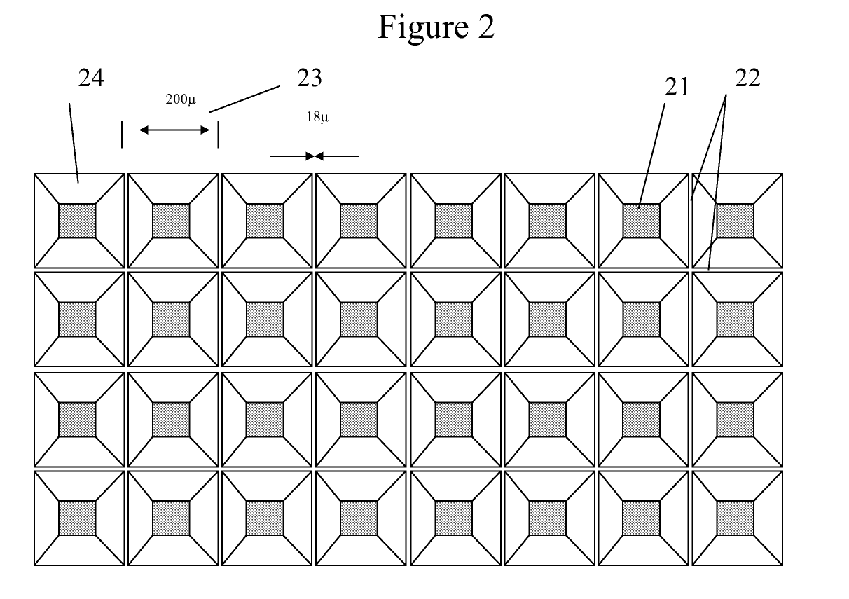

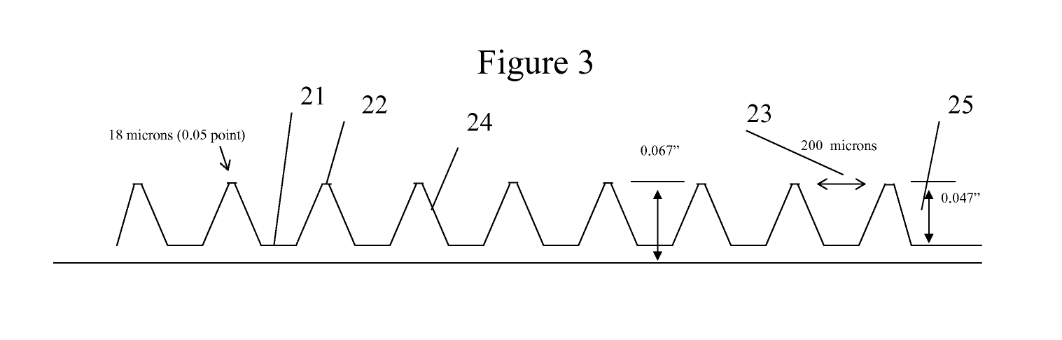

The Cyrel® flexographic printing plates used at both stations were patterned with four 5.1×10.2 cm rectangles. The plates were Cyrel DPL 0.067 inches thick with a floor of 0.019-0.021 inch. The image was a 2″ by 4″ patch containing a crosshatch pattern of 0.05 point rules spaced 200 microns apart. The plates were made on a Dupont Digital Cyrel system using solvent wash. FIG. 2 depicts a portion of the rectangular pattern, showing the microstructure as an array of square depressions, 21, was formed on the plate. The depressions, 21, were separated by a series of perpendicular, 0.05 point lands, spaced 200 micrometers apart, 22, each land being about 18 micrometers wide. The distance between lands being about 200 micrometers, 23, the walls of the depressions, 24, being sloped. The rectangular pattern was oriented 450 to the machine direction of the press. FIG. 3 depicts a cross-sectional view parallel to one side of the rectangle, showing the square depression, 21, the land, 22, the spacing between lands, 23, and the sloped walls of the depressions, 24. The overall thickness of the flexographic plate was 0.670″ (1.7 cm). The wash-out depth was about 0.470″ (1.2 cm).

By weighing a sample of the printed film before and after washing off the scavenger with ethyl acetate, it was found that the 52.0 cm² block of oxygen scavenging ink weighed 0.047 g, or 9.1 g/m². In an oxygen scavenging test as described above, the printed block of scavenger consumed 1.2 mL of oxygen after 24 hours.

### Example 5

Sodium ascorbate powder (Aldrich) and DeniLite® II Base were separately ground by hand in a mortar and pestle. Fifty grams of the thus ground sodium ascorbate and 12.5 grams of the thus ground DeniLite were combined and mixed by shaking. A 2.g portion of the combined powders and 0.40 g of Tween 80 were added to 12.5 g of a 12.5 wt-% solution of EVA in toluene. A portion of this mixture was placed in a 19-mL scintillation vial, three 13 mm×13 mm ceramic cylinders were added, and the vial was rolled for 48 hours. The resulting dispersion was drawn down in a 3×12 cm block on Mylar® film and the oxygen scavenging performance of the coating was tested. The coating weight was 94 mg, or 26 g/m². The sample absorbed 4.8 mL O2 after 64 hours.

### Example 6

2.0 g of calcium ascorbate (Jiangsu Pharma, China. CAS # 5743-28-2) and 0.90 ml of a 5 mg/ml aqueous solution of ascorbate oxidase(Sigma A-0157, CAS 9020-44-1) were combined in 7.0 ml deionized water at room temperature. The resulting solution was frozen and the frozen solution was then placed in a rotary evaporator under vacuum for 24 hours at room temp. The resulting dried mixture was ground into fine powder using a ceramic mortar and pestle. 1.37 g of material were recovered.

To make an ink base, 50 ml ethyl alcohol, 50 ml ethyl acetate and 5 ml Triacetin (Eastman) were combined in a beaker at room temperature. 15 g of cellulose acetate propionate (Eastman CAP482) were added gradually and mixing continued until all CAP had entered solution (several hours). To make ink, 500 mg of the dried calcium ascorbate/ascorbate oxidase mixture powder was added to 5 ml of the ink base.

Two 1.5 ml aliquots of the resulting ink were drawn down onto the surface of a 25 micrometer thick Clairfoil™ cellulose diacetate film (Clarifoil™ North America, Englewood Cliffs, New Jersey). The Clarifoil®″ was clipped to a Diversified Drawdown Platform®. (Diversified Enterprises, 91 N Main St, Claremont, N.H.). In each case, the 1.5 ml aliquot was applied to the Clarifoil®″ using a ½″ diameter Laboratory Metering Rod—Wire Size 30 (Diversified Enterprises) was pulled across the ink and down to the end of the Clarifoil®″, creating a thin layer of film.

The two films so prepared were dried under a nitrogen stream for 60 minutes at room temperature.

The resulting dried ink samples were weighed. The first sample weighed 0.327 g. The second sample weighed 0.419 g.

The oxygen scavenging performance test bottles were incubated for 60 minutes prior to addition of the ink samples. The first sample was placed into the Pyrex® test bottle, and a wet paper towel was placed in the mL scintillation vial. The second sample was placed into the Pyrex® test bottle, and 100 mg of Drierite (W.A. Hammond Drierite Co.) was placed in the 20 mL scintillation vial. The bottles were then sealed. Oxygen scavenging was monitored for 120 hours.

After 120 hours, the atmospheric oxygen concentration In the humidified bottle was observed to have decreased from 20.9% to 17.7%. In the dry bottle, no change in atmospheric oxygen concentration was observed.

### Example 7

A stabilized polymer dispersion was prepared using the procedure of Example 2 in U.S. Pat. No. 5,010,140, with minor modification of some of the monomer components. The polymer was 60.6 wt % of the dispersion and comprised 36% stabilizing shell (hydroxyethyl methacrylate/2-ethylhexyl methacrylate/isobornyl methacrylate/butyl methacrylate; 10/30/15/45) and 64% core (styrene/hydroxyethyl acrylate/methyl methacrylate/glycidyl methacrylate/methacrylic acid; 3.0/20.9/53.4/7.8/0.50/13.9/0.50).

A mixture of 3.0 g of this dispersion, 3.6 g of ethyl acetate, 0.4 g of Tween 80K and 2.0 g of the laccase/calcium ascorbate particles described in Example 1 was mixed for one minute on a Vortex-Genie 2 mixer at a speed setting of 7 and a portion of the suspension was drawn down on Mylar® film. The weight of the 3×12 cm coating was 22 mg, or 61 g/m², and it absorbed 5.3 mL O2 over 24 hours.

### Example 8

This example is directed to a single layer film extrusion coating. The suspension of example 2.1 was drawn down on a 61×23 cm piece of 3-mil Mylar® polyester film (DuPont-Teijin Films) using a #60 Mayer rod. Five blocks scavenger were coated on each 61×23 cm slipsheet. The coated area of each block was limited to a 4.6×12 cm rectangle by using a stencil cut from a second piece of Mylar® film. The coated slipsheets were dried overnight in a vacuum oven at 21° C./22 in. Hg vacuum with a slow nitrogen purge. By weighing a sample of the coating before and after removing the scavenger coating with acetone, it was found that the 55.2 cm² blocks of scavenger had an average coating weight of 20.9 g/m².

The blocks of scavenger were extrusion coated with Elvax® 3200-2 (DuPont) using an Egan pilot coater. The extruder was a 2.5 inch ER-WE-PA 28:1, air cooled. Temperatures were zone 1=270° F., zone 2=360°, zone 3=416°, zone 4=444°, the remainder of the zones and die=450°. The die was a 40″ Cloeren Bead Reduction Die set to a gap of 0.030 inches and deckled to 40″. The chill roll temperature was 50° F. The line speed was 200 feet/min and the extruder speed was adjusted to give a coating weight of 5 lb/ream on 36″ wide 30 lb kraft paper.

A 0.25 inch wide strip of Scotch Adhesive Transfer Tape 924 (3M Industrial Tape and Specialties Division) was applied widthwise to each end of the slipsheet described above, on the back side. The slipsheets were dropped onto the kraft paper web of the extrusion coating so that the transfer tape adhered the slipsheet to the kraft paper substrate, which carried said slipsheet through the coating section of the pilot coater. The coated kraft paper was unwound and the coated slipsheets removed for testing and fabrication.

### Example 9

The example is directed to a two-layer co-extrusion coating. Slipsheets with blocks of scavenger coating were prepared and extruded as described in example 8, except that the film was a bilayer, coextruded film. For the layer against the scavenger blocks, the extruder was a 2.5 inch Egan 24:1 with temperatures: zone 1=358° F., zone 2=448°, zone 3=501°, zone 4=537°, the remainder of the zones and die=536°, melt 552°. The polymer was Bynel® 22E757 (DuPont), applied at 3 lb/ream. For the outer layer, the extruder was a 2.5 inch ER-WE-PA 28:1, air cooled. Temperatures were zone 1=357° F., zone 2=449°, zone 3=503°, zone 4=537°, zone 5=536°, die=450°, melt 536°. The polymer was Appeel® 20D745 (DuPont), applied at 7 lb/ream.

The die was a 40″ Cloeren Bead Reduction Die set to a gap of 0.030 inches and deckled to 40″. The chill roll temperature was 48° F. The line speed was 200 feet/min.

A sample from this coating with a 3×12 cm block of scavenger was tested as described above and found to scavenge 4.4 mL of oxygen in 24 hours. Testing another sample of the coated scavenger with dichlorophenolindophenol (DCPIP) as described above showed that no ascorbate leached through the extrusion coating.

### Example 10

This example is directed to a scavenging film. The suspension of example 3 was drawn down on a 61×23 cm piece of 3-mil Mylar® polyester film (DuPont-Teijin Films) using a #60 Mayer rod. Five blocks scavenger were coated on each 61×23 cm slipsheet. The coated area of each block was limited to a 4.6×12 cm rectangle by using a stencil cut from a second piece of Mylar® film. The coated slipsheets were dried overnight in a vacuum oven at 21° C./22 in. Hg vacuum with a slow nitrogen purge. By weighing a sample of the coating before and after removing the scavenger coating with acetone, it was found that the 55.2 cm² blocks of scavenger had an average coating weight of 20.9 g/m².

The blocks of scavenger were extrusion coated with Elvax® 3200-2 (DuPont) using an Egan pilot coater. The extruder was a 2.5 inch ER-WE-PA 28:1, air cooled. Temperatures were zone 1=270° F., zone 2=360°, zone 3=416°, zone 4=444°, the remainder of the zones and die=450°. The die was a 40″ Cloeren Bead Reduction Die set to a gap of 0.030 inches and deckled to 40″. The chill roll temperature was 50° F. The line speed was 200 feet/min and the extruder speed was adjusted to give a coating weight of 5 lb/ream on 36″ wide 30 lb kraft paper.

A 0.25 inch wide strip of Scotch Adhesive Transfer Tape 924 (3M Industrial Tape and Specialties Division) was applied widthwise to each end of the slipsheet described above, on the back side. The slipsheets were dropped onto the kraft paper web of the extrusion coating so that the transfer tape adhered the slipsheet to the kraft paper substrate, which carried said slipsheet through the coating section of the pilot coater. The coated kraft paper was unwound and the coated slipsheets removed for testing and fabrication.

### Example 11

The example is directed to food package (a pouch) production. Materials: 2 pieces of scavenging film (outer layer(Mylar), scavenger layer from example 10, heat-activated adhesive food contact safe layer. Film pieces were 10 cm×25 cm (scavenger area was 5 cm×14 cm).

Film pieces were arranged with adhesive layers facing each other. Bottom and two sides were sealed using a heat sealer (Kenmore Seal n Save) forming a pouch. A piece of dry tissue paper (Kimwipe) was placed in the bottom of the pouch. A 50 gauge needle was used to poke a hole in one of the sides of the pouch for inserting the oxygen sensor. A plastic cylinder (20 cm long by 0.5 cm diameter) was inserted into the pouch to act as a spacer preventing the two sides from collapsing and sticking to each other.

1.0 ml of H₂O was pipetted directly onto the Kimwipe to provide humidity. The top of the pouch was heat sealed. An oxygen sensor with clipped 50 gauge needle was inserted into the hole created earlier. The gap between the package and the sensor needle was sealed with silicone adhesive (DAP silicone adhesive). Scavenging was monitored at room temp for 24 hours. Internal volume of the package was 28 ml.

The result: Oxygen level within the package was reduced by 1% (20.9 to 19.9) over 24 hours.

### Example 12

Calcium ascorbate/laccase powder was produced by spray drying a 25% by weight solution of calcium ascorbate in water containing 0.25% laccase enzyme. Spray drying was done in a 3 ft diameter, 15 ft³ volume, pilot spray dryer. The dryer was supplied with drying air heated to 228° C. A peristaltic pump was used to meter feed solutions to the spray-drying nozzle. A Spraying Systems SU4, dual fluid nozzle supplied with 30 psi N₂ was used to spray slurries into the volume of the dryer. 75° C. aerosol discharged the dryer to an 8 ft² bag filter where entrained solids were disengaged from the spent drying gas.

A dispersion of the calcium ascorbate/laccase powder was made by mixing 12.7 kg of spray dried enzyme-ascorbate powder in 6.1 kg of propanol with 4.2 kg of Pegosperse 400 MO. This slurry was then media milled in a Premier HM15 mill using an 80% loading of 1.2 to 1.6 mm ER120S media recirculating until a mean particle size of 3 um was achieved.

The resulting dispersion was used to formulate a printable ink by combining: 6.3 kg of the dispersion with 4.3 kg of propanol, 4.1 kg of propyl acetate, 1.1 kg of CAP 482-20, 0.86 kg of polybutyl methacrylate, 0.20 kg of triacetin and 0.43 kg of adipic acid. These were mixed vigorously using an air driven Cowles blade at high speed for 30 minutes.

TABLE 1

| Ingredient | % of ink | Vendor |
|---|---|---|
| Cellulose Acetate Proprionate CAP 482-20 | 4.25 | Eastman |
| Poly(butylmethacrylate) - Elvacite 2044 | 4.00 | Lucite |
| Calcium Ascorbate | 13.86 | |
| Laccase enzyme (dry) | 0.14 | Novozymes |
| Pegosperse 400 MO | 2.80 | Lonza |

The oxygen scavenger ink prepared above was used in a flexographic press to print blocks of oxygen scavenger on a plastic film. The press was a 10″ Mark Andy Inc system 2200 flexographic printing press with 8 print stations. Only stations one and two, provided with Cyrel® (DuPont) printing plates were used to apply two impressions of the scavenger ink and were fitted with 33 BCM (billion cubic micrometers/square inch) anilox rolls. The scavenger was printed on PVDC-coated 0.5 mil polyester film at a speed of 100′/min (30.5 m/min) in the form of 3.5×5 inch blocks, with a coating areal density of about 0.07 g/55 cm2.

The Cyrel® flexographic printing plates used at both stations were patterned with four 3.5×5 inch rectangles. The plates were Cyrel DPL 0.067 inches thick with a floor of 0.019-0.021 inch. The image was a 3.5×5 inch patch containing a crosshatch pattern of 0.05 point rules spaced 100 microns apart. The plates were made on a Dupont Digital Cyrel system using solvent wash. FIG. 2 depicts a portion of the rectangular pattern, showing a microstructure wherein an array of square depressions 21 was formed on the plate. The depressions 21 were separated by a series of perpendicular, 0.05 point lands 22 spaced 100 micrometers apart, each.

The printed film was extrusion coated with EVA (Elvax® 3200-2, DuPont) using an Egan pilot coater. The extruder was a 2.5 inch ER-WE-PA 28:1, air cooled. Temperatures were zone 1=270° F., zone 2=360°, zone 3=416°, zone 4=444°, the remainder of the zones and die=450°. The die was a 40″ Cloeren Bead Reduction Die set to a gap of 0.030 inches and deckled to 40″. The chill roll temperature was 50° F. The line speed was 200 feet/min and the extruder speed was adjusted to give a coating weight of 5 lb/ream on 36″ wide 30 lb kraft paper.

A 0.25 inch wide strip of Scotch Adhesive Transfer Tape 924 (3M Industrial Tape and Specialties Division) was applied widthwise to each end of the slipsheet described above, on the back side. The slipsheets were dropped onto the kraft paper web of the extrusion coating so that the transfer tape adhered the slipsheet to the kraft paper substrate, which carried said slipsheet through the coating section of the pilot coater. The coated kraft paper was unwound and the coated slipsheets removed for testing and fabrication.

The extrusion-coated film was used as lidding film by heat sealing the extruded EVA layer to preformed polyester trays on an Orics (model VGF 200, Orics Industries Inc., College Point, N.Y.) vacuum tray sealer. The sealed trays had an internal volume of 430 cc.

Moist paper towel was sealed within the trays to provide a humidity source to activate the scavenging reaction. The sealed packages were stored at 4 degrees C. Oxygen was measured with a Checkmate II (PBI-Dansensor Ringsted, Denmark). At 24 hours after sealing, 1.6 cc of oxygen was scavenged, after 200 hours 2.0 cc of oxygen was scavenged.

What is claimed is:

1. A method for preparing an activable oxygen scavenging film comprising:
applying a composition to a surface, wherein the composition comprises a non-aqueous liquid vehicle, a multicopper oxidase enzyme, an oxidizable substrate and an organic binder polymer, wherein the polymer is dissolved or dispersed in the liquid vehicle, wherein the enzyme and the substrate are in particulate form and dispersed in the liquid vehicle; and
evaporating the liquid vehicle.

2. The method of claim 1 further comprising laminating the activable oxygen scavenging film with one or more additional layers to form a multi-layer laminate.

3. The method claim 2 wherein at least one of the additional layers is a polymeric film or sheet.

4. The method of claim 3 wherein the polymeric film or sheet acts as a functional barrier.

5. A method for preparing an oxygen reduced package comprising:
applying a composition to a surface wherein the composition comprises: a non-aqueous liquid vehicle, a multicopper oxidase enzyme, an oxidizable substrate and an organic binder polymer, wherein the polymer is dissolved or dispersed in the liquid vehicle, wherein the enzyme and the substrate are in particulate form and dispersed in the liquid vehicle;
evaporating the liquid vehicle forming an activable oxygen scavenging coated surface;
preparing a package having an interior and an exterior, wherein the interior of the package comprises the activable oxygen scavenging coated surface;
providing a source of water vapor wherein the activable oxygen scavenging coated surface is activated; and
sealing the package.

6. The method of claim 5 further comprising applying a functional barrier permeable to oxygen and water vapor at a location to effectively sequester contents of the package from the activable oxygen scavenging coated surface.

7. The method of claim 1 or 5 wherein the organic binder polymer is water-insoluble.

8. The method of claim 1 or 5 wherein the organic binder polymer is selected from the group consisting of poly(ethylene-co-vinyl acetate), cellulose acetate, cellulose acetate propionate, cellulose acetate butyrate, poly(ethylene-co-vinyl acetate-co-carbon monoxide), poly(butylmethacrylate), and mixtures thereof.

9. The method of claim 1 or 5 wherein the liquid vehicle is selected from the group consisting of ethyl acetate, ethanol, toluene, and tetrahydrofuran.

10. The method of claim 1 or 5 wherein the oxidizable substrate is selected from the group consisting of calcium ascorbate, sodium ascorbate, ascorbic acid, ammonium ascorbate, and mixtures thereof.

11. The method of claim 1 or 5 wherein the enzyme and the substrate in particulate form comprises particles in the range of 1 to 100 micrometers.

12. The method of claim 1 or 5 wherein the enzyme and the substrate in particulate form comprises particles in the range of 1 to 20 micrometers.

13. The method of claim 1 or 5 wherein the multicopper oxidase is laccase or ascorbate oxidase.

14. The method of claim 1 or 5 wherein one part by weight of the enzyme and the substrate in particulate form is combined with 0.05 to 20 parts by weight of the organic polymer.

15. The method of claim 1, 5 or 14 wherein one part by weight of enzyme is combined with 20 to 1000 parts by weight of oxidizable substrate.

16. The method of claim 15 wherein one part by weight of enzyme is combined with 50 to 500 parts by weight of oxidizable substrate.

17. The method of claim 1 wherein the enzyme and the substrate in particulate form is present at a concentration in the range of 0.1 to 33 weight % of the total weight of the composition.

18. The method of claim 5 wherein the source of water vapor is food.

19. The method of claim 5 wherein the surface is the surface of a package insert.
